(19) United States
(12) Patent Application Publication
(10) Pub. No.: US 2009/0136601 A1
(43) Pub. Date: May 28, 2009

(54) ANTI-ITCH FORMULATIONS FOR CANINES & METHODS OF USE THEREOF

(76) Inventor: CYNTHIA K. MORALES, DANA POINT, CA (US)

Correspondence Address: LOZA & LOZA LLP, 305 N. Second Avenue, #127, Upland, CA, 91786-6064, US

(21) Appl. No.: 12/330,680

(22) Filed: Dec. 9, 2008

(51) Int. Cl. A61K 36/752

(52) U.S. Cl. 424/736

(57) ABSTRACT

Anti-itch formulations for canines comprised of natural homeopathic ingredients. The formulation includes a combination of oil extracts and other natural ingredients typically characterized as spices or food enhancers. The formulation can be applied to a canine as an itch deterrent in addition to secondary beneficial effects.

### FIELD OF INVENTION

Anti-itch formulations for canines.

### BACKGROUND OF INVENTION

As many dog owners know, fleas can lead to serious health issues for a dog in addition to being a severe irritant caused by the constant scratching when the fleas bite the dog. Flea bites can lead to skin infections, cause discomfort for a pet, lead to allergic skin reactions, and can even transmit other disease-causing organisms, including tapeworms. Flea populations are difficult to control in view of their ubiquitous habitats, i.e., various mammals and outside areas where mammals live.

Commercially available flea and/or parasite deterrents for dogs include Advantage®, Program®, Revolution® and Frontline®. Such products generally use chemicals classified as acetylcholinesterase inhibitors and carry with them a risk of poisoning for both humans and animals. As a result, many dog owners desire natural homeopathic remedies to treat flea infestations.

### SUMMARY OF THE INVENTION

An anti-itch formulation for canines, comprising: between 1.5 ounces and 4.0 ounces of pure orange extract; between 1.5 ounces and 4.0 ounces of peppermint extract; between 3 and 6 bay leaves; about one tablespoon of granulated garlic (dry); and about one tablespoon of chili pepper (dry) is herein disclosed. The anti-itch formulation may more specifically include about 2.7 ounces of pure orange extract, about 2.7 ounces of peppermint extract, and between 4 and 5 bay leaves. The extracts, bay leaves, garlic and chili pepper may be combined in a bottle with an applicator. The extracts, bay leaves, garlic and chili pepper may form a mixture wherein the mixture is formed by shaking or stirring. The anti-itch formulation may have a liquid-like consistency.

A method of treating a canine subject to itching, comprising: combining a sufficient amount of orange oil extract, peppermint oil extract, bay leaves, granulated garlic and chili power to form a mixture; and applying the mixture to the skin of the canine is herein disclosed. The anti-itch formulation may more specifically include about 2.7 ounces of pure orange extract, about 2.7 ounces of peppermint extract, about 4 or 5 bay leaves 5, about one tablespoon of granulated garlic (dry), and about one tablespoon of chili pepper (dry). The formulation may more specifically be applied to the back of a neck of the canine.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention.

Embodiments of the invention are directed to anti-itch formulations for canines comprised of natural homeopathic ingredients. In one embodiment, the formulation includes a combination of oil extracts and other natural ingredients typically characterized as spices or food enhancers. More particularly, an anti-itch formulation may include pure orange extract, peppermint extract, bay leaves, garlic and chili pepper. Although these ingredients are common in the context of food preparation, the combination of these ingredients (in the quantities hereinafter disclosed) led the inventor to observe unexpected results when used as an anti-itch deterrent for dogs.

In one embodiment, an anti-itch formulation includes between 1.5 ounces and 4.0 ounces of pure orange extract, between 1.5 ounces and 4.0 ounces of peppermint extract, between 3 and 6 bay leaves, about one tablespoon of granulated garlic (dry) and about one tablespoon of chili pepper (dry). In a preferred embodiment, the anti-itch formulation includes about 2.7 ounces of pure orange extract, about 2.7 ounces of peppermint extract, about 4 to 5 bay leaves, about one tablespoon of granulated garlic (dry) and about one tablespoon of chili pepper (dry). The pure orange extract may be a suitable mixture of alcohol, water and oil of orange. Similarly, the pure peppermint extract may be a suitable mixture of alcohol, water and oil of peppermint. The ingredients can be mixed together in a container, such as a bottle with an applicator, and shaken or stirred vigorously. The mixture will have a liquid-like consistency with the dry ingredients dispersed or dissolved throughout.

In one application, the anti-itch formulation as described may be applied to a dog as a flea deterrent or an anti-itch deterrent. For example, the anti-itch formulation may be applied to the dog on the back of the dog's neck, directly to the skin thereon. It may be important to apply the formulation on an area of the dog which cannot be readily accessed by the dog by scratching or biting. In this manner, the formulation will have maximum potency because it is not being removed by scratching or removed by biting and therein ingested. Although the ingredients of the formulation are not toxic to the dog (in the quantities applied) as are certain flea deterrents which include acetylcholinesterase inhibitors, ingestion of the formulation may cause mild irritation to the dog's digestive tract if inadvertently ingested in addition to discomfort in the mouth. Therefore, it is important to apply the formulation as described. In addition to the anti-itch benefits described, the formulation also has secondary effects including emotional stability of the dog. This may be due to that the dog is not infested with fleas and constantly scratching or itching as a result of the infestation.

While certain exemplary embodiments have been described and shown in the accompanying drawings, it is to be understood that such embodiments are merely illustrative of and not restrictive on the broad invention, and that this invention is not be limited to the specific constructions and arrangements shown and described, since various other modifications may occur to those ordinarily skilled in the art.

What is claimed is:

1. An anti-itch formulation for canines, comprising:
between 1.5 ounces and 4.0 ounces of pure orange extract;
between 1.5 ounces and 4.0 ounces of peppermint extract;
between 3 and 6 bay leaves;
about one tablespoon of granulated garlic (dry); and
about one tablespoon of chili pepper (dry).

2. The anti-itch formulation of claim 1 wherein the pure orange extract is about 2.7 ounces, the peppermint extract is about 2.7 ounces and the number of bay leaves numbers between 4 and 5.

3. The anti-itch formulation of claim 2 wherein the extracts, bay leaves, garlic and chili pepper are combined in a bottle with an applicator.

4. The anti-itch formulation of claim 3 wherein the extracts, bay leaves, garlic and chili pepper form a mixture, the mixture formed by shaking or stirring.

5. The anti-itch formulation of claim 4 wherein the formulation has a liquid-like consistency.

6. A method of treating a canine subject to itching, comprising:
combining an appropriate amount of orange oil extract, peppermint oil extract, bay leaves, granulated garlic and chili power to form a mixture; and
applying the mixture to the skin of the canine.

7. The method of claim 6 wherein the pure orange extract is about 2.7 ounces, the peppermint extract is about 2.7 ounces, the number of bay leaves numbers between 4 and 5, about one tablespoon of granulated garlic (dry), and about one tablespoon of chili pepper (dry).

8. The method of claim 7 wherein the formulation is applied to the back of a neck of the canine.
